(19) United States
(12) Patent Application Publication
(10) Pub. No.: US 2009/0093795 A1
(43) Pub. Date: Apr. 9, 2009

(54) Peritoneal catheter

(76) Inventor: David William Koeper, Neenah, WI (US)

Correspondence Address: DAVID KOEPER, 3455 GRAND MEADOWS CROSSING, NEENAH, WI, 54956, US

(21) Appl. No.: 11/906,888

(22) Filed: Oct. 4, 2007

(51) Int. Cl. A61M 25/14

(52) U.S. Cl. 604/523

(57) ABSTRACT

A catheter having a first portion having a seal extending from the outer surface thereof and a first hollow-bore diameter, a second portion in fluid communication with the first portion via a venturi, the second portion having a second hollow-bore diameter, and the first hollow-bore diameter is no greater than 90 percent of the second hollow-bore diameter.

### TECHNICAL FIELD OF THE INVENTION

This invention relates to catheters useful in extracting fluids from the peritoneal cavity of humans.

### BACKGROUND OF THE INVENTION

Catheters and suitable for removing fluids from the peritoneal cavity are described in U.S. Pat. No. 4,128,173, issued to Lazarus and Nelson, and U.S. Pat. No. 3,459,188 to Roberts, for example.

These prior catheters have sidewall openings which communicate with a central or axial bore to permit fluids external to the catheter, such as fluids within the abdominal cavity, to be drawn through the sidewall openings into the central bore and thereafter withdrawn from one end of the catheter which is external to the abdominal cavity. Often, the sidewall openings of prior catheters are 0.01 inch to 0.025 inch in diameter, as disclosed in Roberts.

Previous catheters work well except when the sidewall openings become clogged with tissue, as may happen when a catheter is inserted through the peritoneum, for example, or when tissue is drawn into a sidewall opening. When this occurs, the tissue can merely clog the sidewall opening into which the tissue was drawn, or if larger sidewall openings are used, the tissue may pass into the tube itself where, in conjunction with other tissue drawn into the tube, it may create a clog within the tube. The exact mechanism of clogging of larger sidewall openings is not exactly understood, but it seems to involve surface tissue (e.g., of the omentum) being drawn into the sidewall opening during withdrawal or extraction of fluids through the catheter. This surface tissue then remains in the sidewall opening even when fluids are introduced through the catheter into the peritoneum or other cavity.

### SUMMARY AND OBJECTS OF THE INVENTION

The catheter of the present invention comprises a thin-walled, hollow-bore tubular member having a first end, the tubular member having a first portion extending from the first end, the first portion having a first hollow-bore diameter, thereby defining a first hollow-bore cross-sectional area. The catheter further includes a second portion in fluid communication with the first portion, the second portion having a second hollow-bore diameter, thereby defining a second hollow-bore cross-sectional area, and a seal extending from an outer surface of the first portion opposite said first end thereof. The first hollow-bore diameter is no greater than 90 percent of said second hollow-bore diameter.

The invention described herein is particularly effective in improving the non-clogging character of peritoneal catheters, or other catheters in which the external surface of the catheter may be in contact with tissue that may be drawn into the sidewall openings.

It is an object of the instant invention to provide a non-clogging catheter.

It is another object of the instant invention to provide a non-plugging catheter which is easily inserted and removed through tissue.

A further object of the instant invention is to provide a catheter that minimizes injury of the tissue which it passes through.

Another object of the instant invention is to provide catheters with flexible, porous sidewalls.

In accordance with these and other objects, which will become apparent hereinafter, the instant invention will now be described with particular reference to the accompanying drawings.

### BRIEF DESCRIPTION OF DRAWINGS

FIG. 1 is an elevational side view of the preferred embodiment of a catheter of the present invention.

FIG. 2 is an enlargement of the third portion shown in FIG. 1 taken along line 2-2.

FIG. 3 is a cross-sectional view of the second portion of the tubular member folded in upon itself.

FIG. 4 is an elevational side view of a first alternate embodiment of the present invention.

FIG. 5 is an elevational side view of a second alternate embodiment of the present invention.

FIG. 6 is an elevational side view of a third alternate embodiment of the present invention.

### DETAILED DESCRIPTION OF THE PREFERRED EMBODIMENT

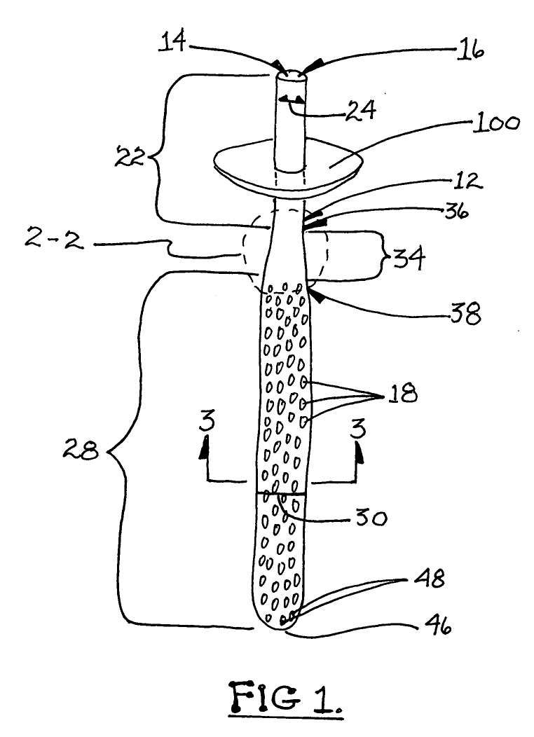

The present invention is particularly useful in peritoneal lavage and dialysis techniques. The preferred embodiment of a catheter 10 of the present invention with non-clogging characteristics is shown in FIG. 1. The catheter 10 comprises a hollow tubular member 12 with a generally circular cross-section, and an opening 14 on at least one end 16. Very fine sidewall openings 18 are spaced circumferentially and longitudinally along the tubular member 12.

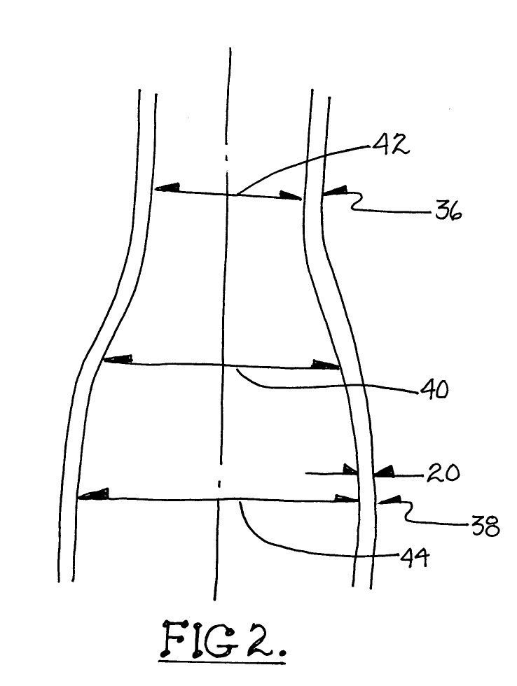

The catheter 10 preferably has a length of about 100 to 250 millimeters, although the actual length will vary based on the application of the catheter 10. The wall thickness 20 of the catheter 10, as shown in FIG. 2, is preferably about 0.5 to 1 millimeter, but for the present invention it is desirable to use a wall thickness 20 that does not result in the catheter 10 being too rigid to fold in upon itself, as described below.

Referring back to FIG. 1, in its preferred embodiment, the catheter 10 of the present invention has a thin-walled, hollow-bore tubular member having a first end 16, and a first portion 22 of the tubular member extends from the first end 16. The first portion 22 has a first hollow-bore diameter 24, which thereby defines a first hollow-bore cross-sectional area. The cross-sectional area of the first portion 22 of the tubular member 12 is preferably constant along the length of the first portion 22.

The preferred embodiment of the present invention further includes a second portion 28 of the tubular member 12 in fluid communication with the first portion 22. The second portion 28 has a second hollow-bore diameter 30, which thereby defines a second hollow-bore cross-sectional area. The cross-sectional area of the second portion 28 of the tubular member 12 is preferably constant along the length of the second portion 28, except at the end 46 where it may be rounded to form a perforated end. The second hollow-core diameter 30 is significantly larger than the first hollow-core diameter 24, such that the first hollow-bore diameter 24 is no greater than 90 percent of said second hollow-bore diameter 30.

The tubular member 12 further includes a third portion 34 connected to the first portion 22 at a first location 36 and connected to the second portion 28 at a second location 38, as shown in FIGS. 1&2. The third portion 34 includes a tapering hollow-bore diameter 40. At the first location 36, the tapering hollow-bore diameter 42 is equal to the first hollow-bore diameter 24, and at the second location the tapering hollow-bore diameter 44 is equal to the second hollow-bore diameter 30. As those skilled in the art will readily appreciate, the tapering hollow-bore diameter 40 forms a venturi that causes an increase in the velocity of fluid flowing from the second portion 28 into the first portion 22 of the tubular member 12.

Referring back to FIG. 1, the tubular member also includes a seal 100 extending from an outer surface of the first portion 22 opposite the first end 16 thereof. As those skilled in the art will readily appreciate, the seal 100 is used to attach the catheter to the patient's skin after the second and third portions 28, 34 have been inserted into the patient's peritoneal cavity. The second portion 28 of the tubular member 12, includes a plurality of sidewall openings 18, and each of the sidewall openings 18 has an opening cross-sectional area. The opening cross-sectional areas may all be formed to be substantially the same (to reduce manufacturing costs), or the opening cross-sectional areas may be varied to preferentially draw more fluid through certain of the sidewall openings 18. Generally, the sidewall openings 18 are round or circular in shape, although oblong or other shapes of sidewall openings 18 may be used. Each of the sidewall opening cross-sectional areas is substantially smaller than the second hollow-bore cross-sectional area 32, and if round, preferably each of the sidewall openings 18 has a diameter of about 0.05 millimeter to about 2.0 millimeters. To ensure that the velocity of fluid flowing through the third portion 34 of the tubular member 12 remains adequate to avoid clogging the tapering hollow-bore 40 of the third portion 34, the sum of the opening cross-sectional areas (i.e. the total flow area provided by the sidewall openings 18) in the second portion 28 must be greater than the second hollow-bore cross-sectional area. The end 46 of the second portion 28 most distant from the third portion 34 is preferably closed, but may have small end openings 48 to allow for fluid flow into the second portion 28 in a manner similar to the fluid flow allowed for by the sidewall openings 18. However, any end openings 48 should be no larger than the largest sidewall openings 18 to prevent tissue large enough to clog the venturi of the third portion 34 from passing into the second portion 28.

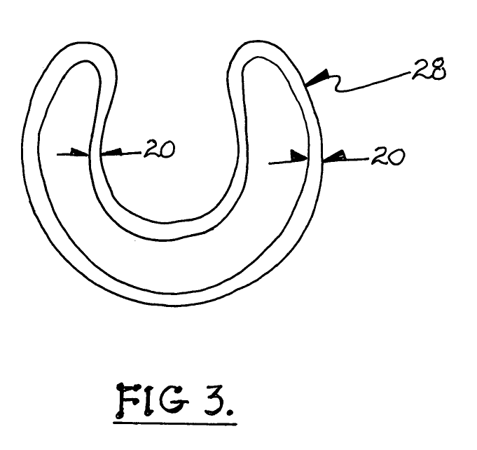

The present invention is preferably made of a plastic or polymeric material that is flexible enough to be deformed by the human hand. By using such a material, the second portion 28, and preferably the third portion 34, of the catheter 10 of the present invention can be folded in upon itself, as shown in cross-section in FIG. 3, prior to and during insertion of the second and third portions 28, 34 into the peritoneal cavity, thereby reducing the necessary size of the abdominal incision in the patient to well below that which would necessary if the second portion 28 of the tubular member 12 were rigid.

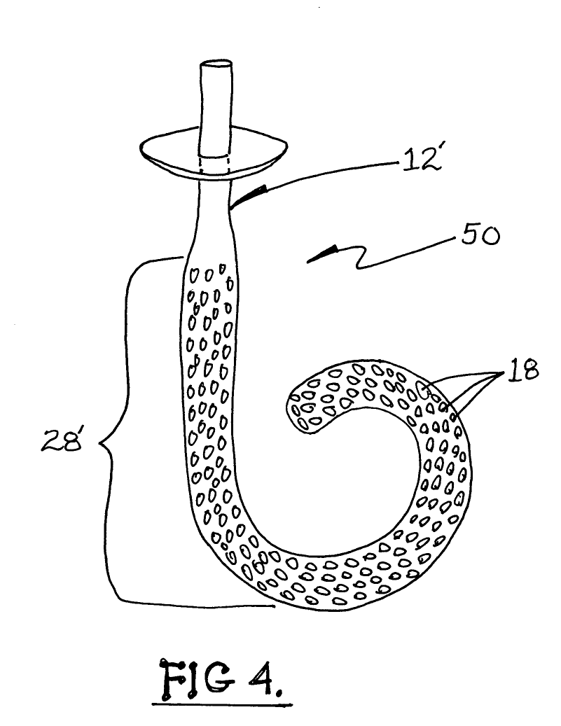

A first alternate embodiment 50 of the present invention, as shown in FIG. 4, is similar to the preferred embodiment, except that the second portion 28′ of the tubular member 12′ is coiled to allow for a substantially longer second portion 28′ that can be inserted into the peritoneal cavity. As those skilled in the art will readily appreciate, coiling the second portion 28′ provides additional sidewall area for inclusion of additional sidewall openings 18, further reducing the likelihood that the entire catheter 50 will become clogged.

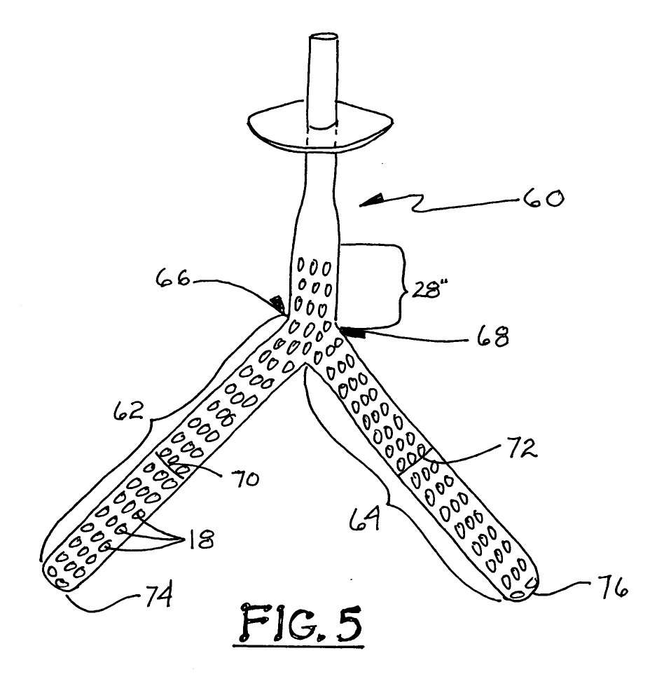

A second alternate embodiment 60 of the present invention, as shown in FIG. 5, is similar to the preferred embodiment, except that the second portion 28″ is preferably shorter, and the second alternate embodiment includes a fourth portion 62 connected to the second portion 28″ at a fourth location 66, and a fifth portion 64 connected to the second portion 28″ at a fifth location 68. Although the second portion 28″ is shown in FIG. 5 as having sidewall openings 18 similar to those in the preferred embodiment, the large flow area provided by the additional sidewall openings in the fourth portion 62 and the fifth portion 64 make the sidewall openings 18 in the second portion 28″ optional.

The fourth portion 62, like the second portion 28″, is tubular and has a fourth hollow-bore diameter 70, thereby defining a fourth hollow-bore cross-sectional area. The fifth portion 64 is likewise tubular and has a fifth hollow-bore diameter 72, thereby defining a fifth hollow-bore cross-sectional area. As shown in FIG. 5, the fourth portion 62 and the fifth portion 64 each extend away from said second portion 28″, the fourth portion 62 has a fourth end 74, the fifth portion 64 has a fifth end 76, and the fourth portion 62 and the fifth portion 64 include a plurality of sidewall openings 18, similar to those described in the preferred embodiment of the invention. As those skilled in the art will readily appreciate, the fourth portion 62 and the fifth portion 64 provide additional sidewall area for inclusion of additional sidewall openings 18, further reducing the likelihood that the entire catheter will become clogged. The fourth portion 62 and the fifth portion 64 are preferably made of a plastic or polymeric material similar to that described in the preferred embodiment.

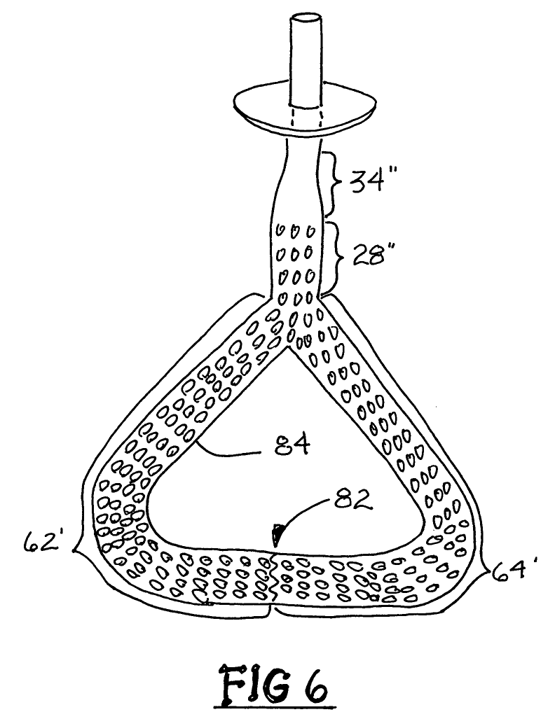

A third alternate embodiment 80 of the present invention, as shown in FIG. 6, is similar to the second alternate embodiment 60, except that the fourth portion 62′ is connected to the fifth portion 64′ at a sixth location 82, thereby forming a loop 84. This loop 84 can be folded onto itself to form a “lead end” for insertion into the peritoneal cavity, followed by the remainder of the fourth and fifth portions 62′, 64′, then the second portion 28″, and then the third portion 34″. As those skilled in the art will readily appreciate, this improvement to the second alternate embodiment 60 eliminates the need to feed the fourth end 74 and the fifth end 76 into the peritoneal cavity separately, which might otherwise require a larger abdominal incision than the incision required for insertion of the third alternate embodiment 80. Although the fourth portion 62′ and the fifth portion 64′ are shown in FIG. 6 as forming a somewhat triangular loop 84, it is to be understood that since the loop is flexible, there are a wide variety of loop shapes that could be utilized in the present invention (e.g. rectangular, circular, oblong, etc.), and therefore the third alternate embodiment of the present invention is not limited to the somewhat triangular shaped loop 84 shown in FIG. 6.

While the invention has been described in what is known as presently the preferred embodiment, it is to be understood that the invention is not to be limited to the disclosed embodiment but, on the contrary, is intended to cover various modifications and equivalent arrangements within the scope of the following claims.

I claim:

1. A catheter comprising:
a thin-walled, hollow-bore tubular member having a first end, said tubular member having
a first portion extending from said first end, said first portion having a first hollow-bore diameter, thereby defining a first hollow-bore cross-sectional area,
a second portion in fluid communication with said first portion, said second portion having a second hollow-bore diameter, thereby defining a second hollow-bore cross-sectional area, and
a seal extending from an outer surface of said first portion opposite said first end thereof,
wherein said first hollow-bore diameter is no greater than 90 percent of said second hollow-bore diameter.

2. The catheter of claim 1 said tubular member further comprises a third portion connected to said first portion at a first location, and connected to said second portion at a second location, said third portion including a tapering hollow-bore diameter that is equal to said first hollow-bore diameter at said first location, and is equal to said second hollow-bore diameter at said second location, wherein said seal is located between said first end and said third portion.

3. The catheter of claim 2 wherein said tapering hollow-bore diameter forms a venturi that causes an increase in the velocity of fluid flowing from the second portion into the first portion of the tubular member.

4. The catheter of claim 3 wherein said second portion further comprises a plurality of sidewall openings in said second portion, each of said openings having an opening cross-sectional area, wherein the sum of said opening cross-sectional areas is greater than said second hollow-bore cross-sectional area.

5. The catheter of claim 4 wherein each of said opening cross-sectional areas is substantially smaller than said second hollow-bore cross-sectional area.

6. The catheter of claim 5 wherein each of said sidewall openings has a diameter of from about 0.01 to about 2.0 millimeters.

7. The catheter of claim 3 further comprising a fourth portion connected to said second portion at a fourth location, and a fifth portion connected to said second portion at a fifth location, said fourth portion having a fourth hollow-bore diameter, thereby defining a fourth hollow-bore cross-sectional area, said fifth portion having a fifth hollow-bore diameter, thereby defining a fifth hollow-bore cross-sectional area, wherein said fourth portion and said fifth portion each extend away from said second portion.

8. The catheter of claim 7 wherein said fourth portion and said fifth portion include a plurality of sidewall openings, each of said openings having an opening cross-sectional area, wherein the sum of said opening cross-sectional areas is greater than said second hollow-bore cross-sectional area.

9. The catheter of claim 8 wherein each of said opening cross-sectional areas is substantially smaller than said second hollow-bore cross-sectional area.

10. The catheter of claim 9 wherein each of said sidewall openings has a diameter of from about 0.01 to about 2.0 millimeters.

11. The catheter of claim 7 wherein said second portion, said fourth portion and said fifth portion include a plurality of sidewall openings, each of said openings having an opening cross-sectional area, wherein the sum of said opening cross-sectional areas is greater than said second hollow-bore cross-sectional area.

12. The catheter of claim 11 wherein each of said opening cross-sectional areas is substantially smaller than said second hollow-bore cross-sectional area.
